(19) United States
(12) Patent Application Publication
(10) Pub. No.: US 2025/0062023 A1
(43) Pub. Date: Feb. 20, 2025

(54) MACHINE LEARNING METHODS AND SYSTEMS FOR PHENOTYPE CLASSIFICATIONS

(71) Applicant: Plan Heal Health Companies, Inc., Chicago, IL (US)

(72) Inventors: Tammy McMiller, Chicago, IL (US); Eric A. McMiller, Chicago, IL (US); Luke Paul McMiller, Chicago, IL (US)

(21) Appl. No.: 18/720,358

(22) PCT Filed: Dec. 5, 2022
(86) PCT No.: PCT/US2022/051772

§ 371 Date: Jun. 14, 2024

Related U.S. Application Data
(60) Provisional application No. 63/290,323, filed on Dec. 16, 2021.

(51) Int. Cl. G16H 50/20; G16H 20/60; G16H 50/30

(52) U.S. Cl. CPC G16H 50/20; G16H 20/60; G16H 50/30

(57) ABSTRACT

Methods and computing apparatus for implementing machine learning models for phenotype classifications. A machine-learned model is trained based on a data classification path process that includes obtaining patient data, identifying classification results, determining patient data classification path features, and selecting patient data classification path features for inclusion in the machine-learned patient data classification path process using a sequencing protocol that defines a minimal causal relationship that exists between a particular patient data classification path feature and identified patterns. A path classification request that includes a first set patient data elements associated with a particular patient for a first time period is received from a user device. A plurality of path classification outcomes associated with the particular patient based on the patient data elements is determined. A unique phenotype classification associated with the particular patient for the first time period based on the plurality of path classification outcomes is determined.

### TECHNICAL FIELD

The present invention generally relates to computers and computer software, and more specifically, to methods, systems, and computer program products for implementing machine learning models for phenotype classifications.

### BACKGROUND

Machine learning is increasingly prevalent in and vital to health care industries in terms of predicting and identifying quality treatments for patients and enhancing other health care services. Machine learning techniques are used for extracting knowledge from large and complex data sets in an organized form in order to make more effective decisions. Additionally, because of the increasing amount of available data, machine learning techniques have significant benefits as prediction tools in health care that sometimes provide surprising prediction models that help in clinical counseling. These tools are fundamental to biomedical research and are utilized as an integral part of the clinical decision-making process.

For example, in some instances, patients may or may not know they have a particular disease, and they can go years without being diagnosed. Because of this, there may be other interrelated diseases that could occur as a result of the initial disease. Thus, it would be desirable to have a time sequence protocol that automates a year-over-year monitoring of a patient to help their medical practitioner deliver the right protocols for risk and care at the right time.

### SUMMARY

In embodiments of the invention, a method for implementing a phenotype classification process. The method includes, at an electronic device having a processor, training a machine-learned model based on a patient data classification path process for each iteration of a plurality of iterations. Training the machine-learned model may include obtaining patient data stored within a patient database, wherein the patient database is populated with a plurality of patient data elements associated with one or more patients. Training the machine-learned model may further include evaluating the patient data elements to determine and identify classification results based on predetermined classification database tables. Training the machine-learned model may further include determining patient data classification path features based on the identified classification results. Training the machine-learned model may further include selecting one or more of the patient data classification path features for inclusion in the machine-learned patient data classification path process using a sequencing protocol that defines a minimal causal relationship that exists between a particular patient data classification path feature and identified patterns. The method may further include receiving a phenotype classification request from a user device, the path classification request including a first set patient data elements associated with a particular patient for a first time period. The method may further include determining, utilizing the machine-learned patient data classification path process, a plurality of path classification outcomes associated with the particular patient based on the patient data elements. The method may further include determining, utilizing the machine-learned patient data classification path process, a unique phenotype classification associated with the particular patient for the first time period based on the plurality of path classification outcomes.

These and other embodiments can each optionally include one or more of the following features.

In some embodiments of the invention, the patient data elements associated with the particular patient includes a first disease that includes an active time window. In some embodiments of the invention, the patient data elements associated with the particular patient includes a type of disease and a date of contraction.

In some embodiments of the invention, the method further includes sending the unique use phenotype classification associated with the particular patient to the user device. In some embodiments of the invention, determining the unique phenotype classification associated with the particular patient for the first time period is based on detecting a disease that is associated with the unique phenotype classification associated with the particular patient.

In some embodiments of the invention, the method further includes receiving a second path classification request from the user device, the second path classification request including a second set of patient data elements associated with the particular patient for a second time period, and determining a second phenotype classification associated with the particular patient for the second time period. In some embodiments of the invention, the first set of patient data elements includes a first disease, and the second set of patient data elements includes a second disease that is different than the first disease, wherein the first disease and second disease include interrelated attributes. In some embodiments of the invention, determining the second phenotype classification associated with the particular patient for the second time period is based on analysis of an active time window associated with the first disease and an active time window associated with the second disease.

In some embodiments of the invention, the second phenotype classification is different than the first phenotype classification.

In some embodiments of the invention, the machine-learned patient data classification path process is based on determining a timeline of risk and detection of disease based on a patient's individual health status. In some embodiments of the invention, the minimal causal relationship exists before that particular patient data classification path feature is included in the machine-learned patient data classification path process.

In embodiments of the invention, a computing apparatus for implementing a phenotype classification process. The computing apparatus includes one or more processors, at least one memory device coupled with the one or more processors, and a data communications interface operably associated with the one or more processors. The at least one memory device contains a plurality of program instructions that, when executed by the one or more processors, cause the including apparatus to perform operations. The operations include training a machine-learned model based on a patient data classification path process for each iteration of a plurality of iterations. Training the machine-learned model may include obtaining patient data stored within a patient database, wherein the patient database is populated with a plurality of patient data elements associated with one or more patients. Training the machine-learned model may further include evaluating the patient data elements to determine and identify classification results based on predetermined classification database tables. Training the machine-learned model may further include determining patient data classification path features based on the identified classification results. Training the machine-learned model may further include selecting one or more of the patient data classification path features for inclusion in the machine-learned patient data classification path process using a sequencing protocol that defines a minimal causal relationship that exists between a particular patient data classification path feature and identified patterns. The operations may further include receiving a phenotype classification request from a user device, the path classification request including a first set patient data elements associated with a particular patient for a first time period. The operations may further include determining, utilizing the machine-learned patient data classification path process, a plurality of path classification outcomes associated with the particular patient based on the patient data elements. The operations may further include determining, utilizing the machine-learned patient data classification path process, a unique phenotype classification associated with the particular patient for the first time period based on the plurality of path classification outcomes.

In embodiments of the invention, a non-transitory computer storage medium encoded with a computer program, the computer program including a plurality of program instructions that when executed by one or more processors cause the one or more processors to perform operations. The operations include training a machine-learned model based on a patient data classification path process for each iteration of a plurality of iterations. Training the machine-learned model may include obtaining patient data stored within a patient database, wherein the patient database is populated with a plurality of patient data elements associated with one or more patients. Training the machine-learned model may further include evaluating the patient data elements to determine and identify classification results based on predetermined classification database tables. Training the machine-learned model may further include determining patient data classification path features based on the identified classification results. Training the machine-learned model may further include selecting one or more of the patient data classification path features for inclusion in the machine-learned patient data classification path process using a sequencing protocol that defines a minimal causal relationship that exists between a particular patient data classification path feature and identified patterns. The operations may further include receiving a phenotype classification request from a user device, the path classification request including a first set patient data elements associated with a particular patient for a first time period. The operations may further include determining, utilizing the machine-learned patient data classification path process, a plurality of path classification outcomes associated with the particular patient based on the patient data elements. The operations may further include determining, utilizing the machine-learned patient data classification path process, a unique phenotype classification associated with the particular patient for the first time period based on the plurality of path classification outcomes.

The above summary may present a simplified overview of some embodiments of the invention in order to provide a basic understanding of certain aspects of the embodiments of the invention discussed herein. The summary is not intended to provide an extensive overview of the embodiments of the invention, nor is it intended to identify any key or critical elements, or delineate the scope of the embodiments of the invention. The sole purpose of the summary is merely to present some concepts in a simplified form as an introduction to the detailed description presented below.

### BRIEF DESCRIPTION OF THE DRAWINGS

The accompanying drawings, which are incorporated in and constitute a part of this specification and in which like reference numerals refer to like features, illustrate various embodiments of the invention and, together with the general description given above and the detailed description given below, serve to explain the embodiments of the invention.

FIG. 1 illustrates an environment for implementing a phenotype classification process using machine learning models, according to embodiments of the invention.

FIG. 2 is a block diagram illustrating a path classification prediction process in accordance with embodiments of the invention.

FIG. 3 shows a flowchart of a method of updating a machine learning model in accordance with embodiments of the invention.

FIG. 4 illustrates example path classification data, according to embodiments of the invention.

FIG. 5 illustrates an example phenotype classification process based on a path classification request, according to embodiments of the invention.

FIG. 6 is a flowchart of an example process for training a machine-learned model based on a patient data classification path process for a plurality of iterations, according to embodiments of the invention.

FIG. 7 is a flowchart of an example process for determining a unique phenotype classification associated with a patient based on a plurality of path classification outcomes, according to embodiments of the invention.

FIG. 8 is a block diagram showing an example computer architecture for a computer capable of executing the software components described herein, according to embodiments described herein.

### DETAILED DESCRIPTION

The technology in this patent application is related to systems and methods for implementing a machine learned phenotype classification path process as a feature in a records database environment of a health system. The phenotype classification process provides a clinician with the possibility of determining a unique patient phenotype classification for a particular patient that can be used for clinical research and/or treatment reference. As the machine learning model acquires more patient data, database data tables increase as more path classification outcomes are added. For example, outcomes are identified at time of detection and identification of risk.

In some implementations of the invention, a machine learned phenotype classification path process may include collection of data sets and supervised correlation of the datasets to determine a classification type of specific disease risk analysis path or contracted disease path. The disease paths may be generated based on automated data collection over a period of time frame to detect manifestation of interrelated disease. Each instance results in the identification of a phenotype using unsupervised machine learning. A unique user phenotype classification may be determined by unsupervised machine learning that's determined by a neural network algorithm that determines the timeline of risk and detection of disease based on the patient's individual health status, e.g., contraction of a disease and interrelated diseases, timeframe of the contraction of a disease, or not contracting the disease at all.

More specifically, this technology includes a process that trains a machine-learned model based on a patient data classification path process for each iteration of a plurality of iterations. First, patient data stored within a patient database is obtained, where the patient database is populated with a plurality of patient data elements associated with a one or more patients. For example, patient's real time data inputs are captured based on data tables that include various population health parameters. Data inputs are collected manually, automatically via devices, and by geolocation. Patient data input prompts are automated based on certain general health and health determinants factors. The patient data elements may include a type of disease and date of contraction. For example, if known, lifestyle factors, other factors including administrative and demographic information, diagnosis, treatment, prescription drugs, laboratory tests, physiologic monitoring data, and the like, may all be included in the analysis. Next, the patient data elements are evaluated to determine and identify classification results based on predetermined classification database tables. For example, database classifications are determined and identified in reference to predetermined classification database tables that include a specific set of diseases. Then, patient data classification path features are determined based on the identified classification results. For example, each path classification has a set of time sequenced data inputs and correlation analysis that triggers real time tracking instances for continuous monitoring, preventing, and detecting disease that is associated with a specific path classification.

In some implementations of the invention, one or more of the patient data classification path features are selected for inclusion in the machine-learned patient data classification path process using a sequencing protocol that defines a minimal causal relationship that exists between a particular patient data classification path feature and identified patterns. For example, the outcomes are correlated to determine a unique patient phenotype classification that's determined by timeline of risk and detection of disease based on the patient's individual health status (e.g., contraction of a disease and interrelated diseases, timeframe of the contraction of a disease, or not contracting the disease at all).

After training the machine learning model for the patient data classification path process, this technology includes a process that initially receives a path classification request from a user device. The path classification request may include a plurality of patient data elements associated with a particular patient for a first time period (e.g., diseases known, time frames/dates, lifestyle factors, etc.). A plurality of path classification outcomes associated with the particular patient may be determined utilizing the machine-learned patient data classification path process and based on the patient data elements. For example, paths classifications data may be correlated in real time and sequenced to determine unique outcomes. A unique phenotype classification associated with the particular patient for the first time period may be determined utilizing the machine-learned patient data classification path process and based on the plurality of path classification outcomes. For example, user phenotype classification is correlated in phenotype classification database for clinical research and/or treatment reference.

Although the examples provided herein reference phenotype classifications within the medical industry, the machine learning processes described may be applied to other complex data systems that include interrelated time sequenced variables.

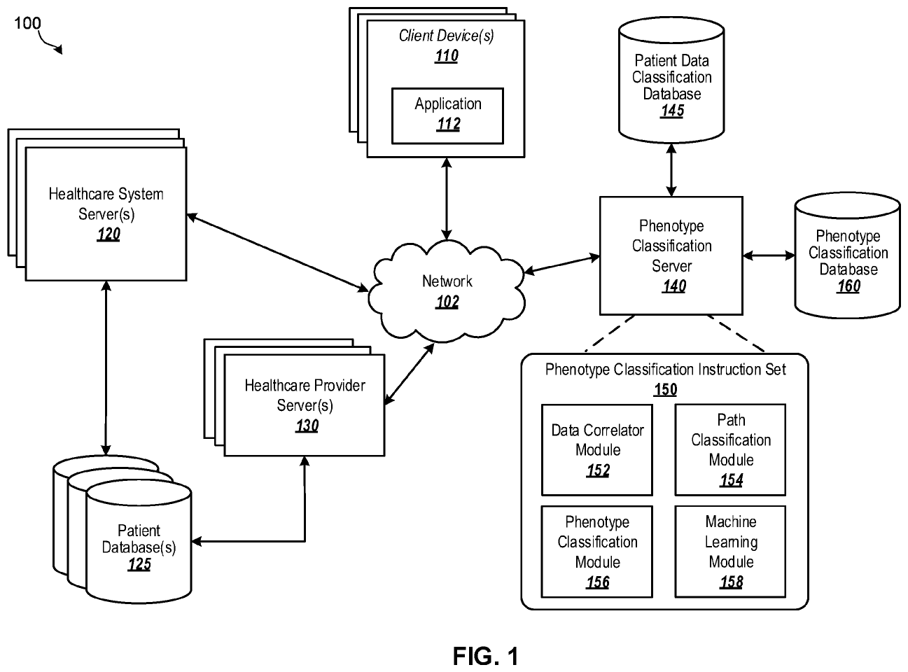

FIG. 1 is an example environment 100 for implementing a phenotype classification process, according to embodiments of the invention. The example environment 100 includes one or more client device(s) 110, one or more healthcare system server(s) 120, one or more healthcare provider server(s) 130, and a phenotype classification server 140, that communicates over a data communication network 102, e.g., a local area network (LAN), a wide area network (WAN), the Internet, a mobile network, or a combination thereof.

The one or more client device(s) 110 (e.g., a device used by a phenotype classification requestor, such as a clinician, clinical researcher, etc.) can include a desktop, a laptop, a server, or a mobile device, such as a smartphone, tablet computer, and/or other types of mobile devices. The one or more client device(s) 110 includes applications, such as the application 112, for managing a classification request to/from the phenotype classification server 140, as well as providing the initial rulesets to the one or more healthcare system server(s) 120. The one or more client device(s) 110 can include other applications. The one or more client device(s) 110 initiates a phenotype classification request by a requestor via application 112. The phenotype classification request may include instructions that include one or more sets of rules setup by the requesting entities (such as clients, applications, browsers installed on user terminals, etc.) in the course of a phenotype classification. The one or more client device(s) 110 may be utilized by a user (e.g., a clinician) to review phenotype classification results.

The one or more healthcare system server(s) 120 are entities such as hospitals, healthcare management, government health services, and the like, that manage system wide healthcare data (e.g., via healthcare compliant protocols). The one or more healthcare provider server(s) 130 are entities such as doctor's offices, clinics, and the like, that manage individual patient data at the point of care for each individual patient. The one or more healthcare system server(s) 120 and/or the one or more healthcare provider server(s) 130 may be a personal computing device, tablet computer, thin client terminal, smart phone and/or other such computing device capable of managing and protecting healthcare data per HIPPA and other government regulated protocols.

The healthcare system server(s) 120 and/or healthcare provider server(s) 130 may access patient data and/or store patient data as patient records in the patient database(s) 125. Each patient record may include a plurality of demographic attributes associated with the patient, such as the first, middle and last name of the person, the mailing address of the person, the date of birth of the person, etc. Additionally, a patient record may include information describing one or more encounters of a patient with a respective healthcare facility. Patient records may include information regarding a wide variety of encounters including office visits, laboratory tests, hospital admittances, imaging appointments, etc. Some patient records may also include or otherwise be associated with one or more documents. The documents may be associated with one or more of the encounters for which the patient record includes information. The documents may include, for example, laboratory results, notes taken by a physician during an office visit, imaging studies or the like.

The phenotype classification server 140 receives and processes the classification request(s) from a client device 110. The phenotype classification server 140 may be a personal computing device, tablet computer, thin client terminal, smart phone and/or other such computing device. The phenotype classification server 140 includes a phenotype classification instruction set 150 that performs a path classification protocol according to processes described herein.

The phenotype classification instruction set 150 may include a data correlator module 152 for correlating the patient data. For example, data correlation may include analyzing the patient records for patient's real time data inputs that are captured based on data tables that include various population health parameters. Data inputs are collected manually, automatically via devices, and by geolocation. Patient data input prompts may be automated based on certain general health and health determinants factors. The patient data elements may include a type of disease and date of contraction (if known, lifestyle factors, other factors including administrative and demographic information, diagnosis, treatment, prescription drugs, laboratory tests, physiologic monitoring data).

In some implementations of the invention, the phenotype classification instruction set 150 further includes a path classification module 154 for evaluating the patient data elements to determine and identify classification results based on predetermined classification database tables based on the received correlated data from the data correlator module 152. In some implementations of the invention, the path classification module 154 may determine patient data classification path features based on the identified classification results. For example, classification results determine paths, and each path classification may have a set of time sequenced data inputs and correlation analysis that trigger real time tracking instances for continuous monitoring, preventing, and detecting diseases that may be associated with a specific path classification. Additionally, within each path classification there may be a specific set of unique timed instances. Each path classification that is identified is then stored by path classification module 154 into the patient data classification database 145.

In some implementations of the invention, the phenotype classification instruction set 150 further includes a phenotype classification module 156 for managing phenotype classifications. In some implementations of the invention, the phenotype classification module 156 may be utilized in the process of selecting one or more of the patient data classification path features for inclusion in a machine-learned patient data classification path process using a sequencing protocol that defines a minimal causal relationship that exists between a particular patient data classification path feature and identified patterns. For example, the outcomes of a patient classification may be correlated to determine a unique user phenotype classification that's determined by a timeline of risk and detection of a disease based on the patient's individual health status (e.g., contraction of a disease and interrelated diseases, timeframe of the contraction of a disease, or not contracting the disease at all, and the like). Each phenotype classification that is identified is then stored by phenotype classification module 156 into the phenotype classification database 160.

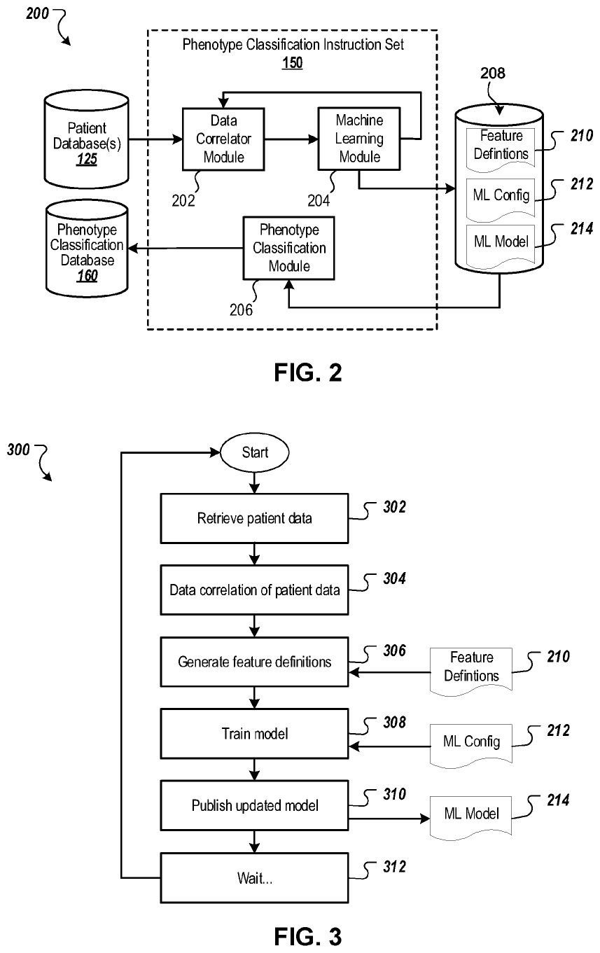

In order to achieve quality decision-making at high speed in the context path classifications and phenotype classifications, embodiments of the present invention employ a machine learning approach. For example, in some implementations of the invention, the phenotype classification instruction set 150 further includes a machine learning module 158 which is configured to process raw data relating to patient data, to generate training data sets for a machine learning model, and to train the machine learning model for deployment to the phenotype classification server 140. The processing, training, and deployment actions are described in greater detail below, with reference to FIGS. 2 and 3, and may be carried out continuously, periodically and/or on-demand in order to maintain currency of the machine learning model.

FIG. 2 is a block diagram illustrating schematically a number of code modules that together include a path classification prediction engine 200 embodying the invention. Implementation of the path classification prediction engine 200 is distributed within the machine learning module 158 of the phenotype classification server 140. Two code modules make up the server component of the engine 200, namely a data correlator module 202 and a machine learning module 204. In some implementations of the invention, additional code modules may be utilized (e.g., a feature enrichment module, and the like). These two (or more) modules are implemented within the program instructions of the phenotype classification instruction set 150 executing on the phenotype classification server 140. The functionality implemented within each of these modules will now be described in greater detail.

The purpose of the data correlator module 202 (e.g., data correlator module 152 of FIG. 1) is to correlate the received patient data. For example, data correlation may include analyzing the patient records for patient's real time data inputs that are captured based on data tables that include various population health parameters. Data inputs are collected manually, automatically via devices, and by geolocation. Patient data input prompts may be automated based on certain general health and health determinants factors. The patient data elements may include a type of disease and date of contraction (if known, lifestyle factors, other factors including administrative and demographic information, diagnosis, treatment, prescription drugs, laboratory tests, physiologic monitoring data).

The general approach employed for data correlation in embodiments of the invention is to identify, in the patient data, particular health element or events and subsequent interaction events within a predetermined time window that have a selected set of parameters. The time window should be of sufficient duration to capture a substantial majority of all interactions, and the number and choice of parameters should be sufficient to ensure unique correlation in a substantial majority of cases. Perfect correlation may be difficult to achieve, because it is impossible to know if or when an interaction will occur. The risk of erroneous correlation can be reduced by using a larger selected set of parameters to distinguish between different sets of healthcare elements (e.g., diseases known, time frames/dates, lifestyle factors, etc.), at the expense of making the correlation process more complex.

In exemplary experimental embodiment, the invention has been implemented in the context of a domain-specific phenotype classification server 140 operating on behalf of healthcare providers, using patient data captured from a live system (e.g., patient database(s) 125). A heuristic approach was taken to design of the correlation module, with a number of experiments being conducted to determine a suitable time window, and a selected set of parameters. The following event parameters were found to be effective with correlation: diseases known, time frames/dates, lifestyle factors, etc. In the exemplary embodiment, correlation is performed using neural network algorithms to deliver unsupervised machine learning that is trained to recognize patterns in order to cluster data inputs for classification using dimensionality reduction.

A feature enrichment module may be included to derive, from the values of raw features in the correlated data generated by the data correlator module 202, a corresponding set of enriched feature vectors for use by the machine learning module 204. Definitions of features for use by the feature enrichment module 204 are shown as being stored in a file 210 within data store 208, however this may be regarded as a schematic convenience. In a practical embodiment, feature definitions may be stored in this way, may be compiled into a code module and linked to the feature enrichment module, or may be hard-coded into the feature enrichment module. As will be appreciated, each of these implementation options potentially offers a different trade-off between flexibility, code complexity, and execution speed.

The machine learning module 204 includes a program code executing on the phenotype classification server 140, and configured in the exemplary experimental embodiment to implement a generalized linear model. Specifically, the machine learning module 204 of the exemplary embodiment implements a regularized logistic regression algorithm, with ‘follow-the-regularized-leader’ (FTRL)-proximal learning. The algorithm has a number of hyperparameters that can be adjusted in order to optimize its learning accuracy on the training data for a specific problem. In FIG. 2, fixed values of the hyperparameters for use by the machine learning module 204 are shown as being stored in a file 212 within data store 208. As will be appreciated, however, alternative implementations are possible, such as hard-coding the parameters into the machine learning module 204.

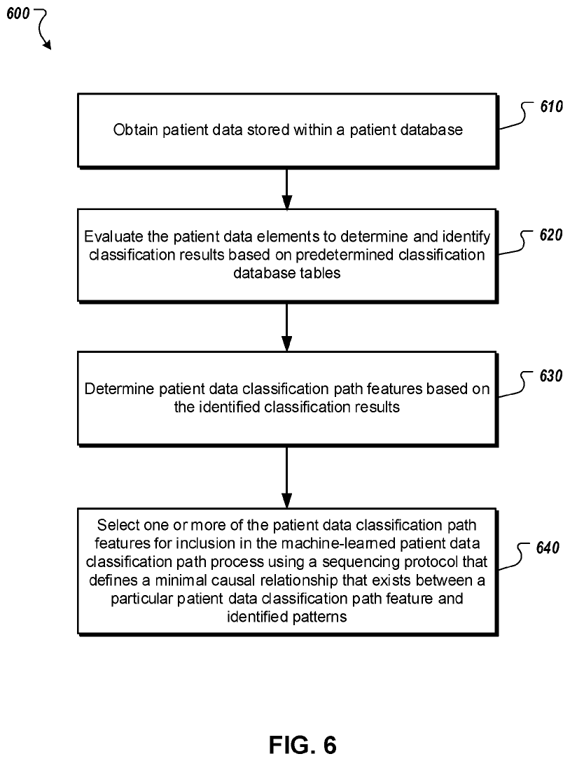

Execution of the machine learning module 204 on a particular patient dataset results in the generation of a model that can be executed by the phenotype classification instruction set 150 of the phenotype classification server 140, as will be described in greater detail below with reference to process 600 of FIG. 6. In use, the phenotype classification server 140 executes the modules 202 and 204 repeatedly, e.g., continuously, periodically, or on-demand. This is illustrated by the flowchart 300 shown in FIG. 3.

The path classification prediction engine 200 further includes a phenotype classification module 206, which is implemented within the phenotype classification instructions 150 executing on the phenotype classification server 140. The phenotype classification module 206 employs the feature definitions 210 and the trained model representation 214. Phenotype classification module 206 predicts phenotype classifications for a particular patient, and stores the phenotype classifications in the phenotype classification database 160. Phenotype classification module 206 may predict phenotype classifications using neural network algorithms to deliver unsupervised machine learning that is trained to recognize patterns in order to cluster data inputs for classification using dimensionality reduction.

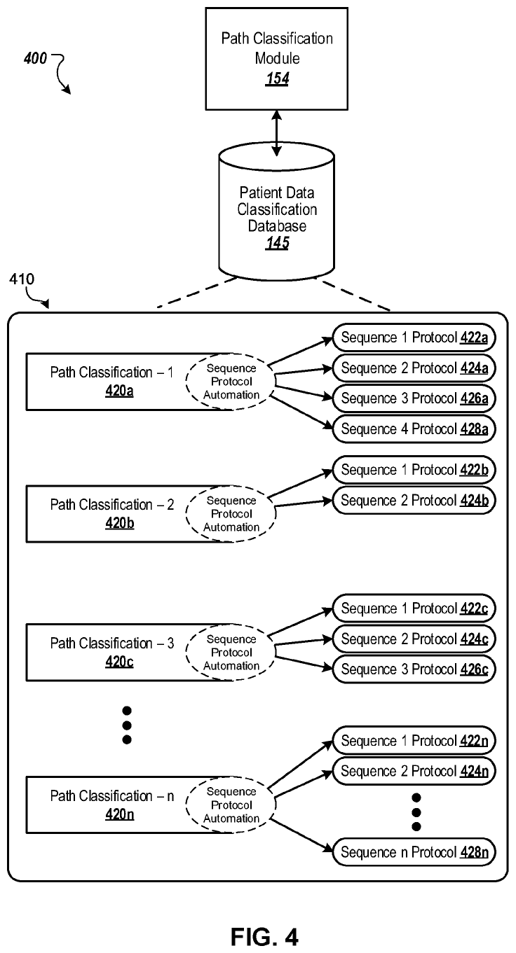

In some embodiments of the invention, the engine 200 includes a path classification module that, similarly to the phenotype classification module 206, predicts path classifications, and stores the path classifications in the path classification database 145. An example illustration of path classifications is further described herein with reference to FIG. 4.

FIG. 3 illustrates an example phenotype classification process based on patient data, according to embodiments of the invention. Patient data is retrieved from the patient database(s) 125 at block 302. At block 304, the correlation module (e.g., data correlator module 202) performs correlation of patient data, as described. In practice, retrieval block 302 and correlation block 304 may be combined as a single query, e.g., an Impala SQL query.

At block 306, the phenotype classification server 140 executes a feature module (e.g., a feature enrichment module), which uses the feature definitions 210 to compute enriched feature vectors corresponding with the correlated data. These are transferred to the machine learning module 204 which trains the model at block 308 using the feature vectors and the predetermined hyperparameters defined in the configuration file at block 312. The resulting model coefficients are hashed, serialized, and published at block 310 to the model file 214.

Optionally, the phenotype classification server 140 then waits at block 312, before recommencing the process at block 302. Exit from the wait condition at block 312 may be triggered by a number of different events. For example, the phenotype classification server 140 may be configured to run the modules 202 and 204 periodically, e.g., once per day. Alternatively, or additionally, it may be configured to run the modules 202 and 204 on-demand, e.g., upon receipt of a signal from a controller (not shown) within the system 100. In some embodiments the machine learning module 158 of the phenotype classification server 140 may run the modules 202 and 204 continuously, thereby updating the model file 214 as frequently as possible based upon the time required for data correlation, feature enrichment, and model training.

FIG. 4 illustrates example environment 400 regarding automated classification paths, according to embodiments of the invention. In particular, FIG. 4 illustrates example path sequence protocols for different potential path classifications as stored and classified in the patient data classification database 145 by the path classification module 154 of the phenotype classification instruction set 150 executed by the phenotype classification server 140. Database structure 410 includes a plurality of path classifications based on machine learning model correlating and classifying several different patients (e.g., obtained via the patient database(s) 125 from various healthcare entities). For example, database structure 410 illustrates example path classifications: path classification—1 420a, path classification—2 420b, path classification—3 420c, through path classification—n 420n (also referred to as a path classification 420). Each path classification 420 may be sequenced in multiple different sequencing protocols. For example, for illustrative purposes, path classification—1 420a includes a sequence protocol automation that determines four different sequencing protocols: sequence 1 protocol 422a, sequence 2 protocol 424a, sequence 3 protocol 426a, and sequence 4 protocol 428a. Similarly, each path classification 420a-n, may each include one or more sequence protocol.

For example, Path Classification—1 420a may be correlated by: i) if the patient tests positive for a disease when the date of contraction is unknown, or ii) the patient's lifestyle and other factors is a risk for a particular disease, or iii) the patient has had the particular disease treated but is still at risk for other interrelated diseases and/or cancers. Each “or” decision and correlation of data is triggered by a machine learning algorithm to determine what the sequence time for a protocol might be. Additionally, each year may include an updated sequence protocol (e.g., a patient goes in for an annual physical).

In some instances, a patient can be on more than one path classification 420. For example, a patient can be on path classification—1 420a, and path classification—2 420b.

Time Sequence Example for Path Classification—1 420a:
Year 1: Automated protocol to test for disease 1.
Year 2: The patient and doctor received automated protocols based on contraction of disease 1 (this includes a series of protocols ranging from one or more).
Year 4: The patient and doctor receive automated protocols for interrelated disease 1, interrelated disease 2, interrelated disease 3, and interrelated disease 4.
Year 6: The patient and doctor receive automated protocols for interrelated disease 1, and interrelated disease 2 (this can include a series of protocol ranging from one or more).
Year 8: The patient and doctor receive automated protocol for interrelated disease 1 (this includes a series of protocol ranging from one or more).
Year 10: The patient and doctor repeat the protocol for years 4, 6, and 8 over time, and the cycle can repeat to detect and prevent potential diseases (e.g., interelatred diseases) as patients are screened until a particular age (i.e., age 65).

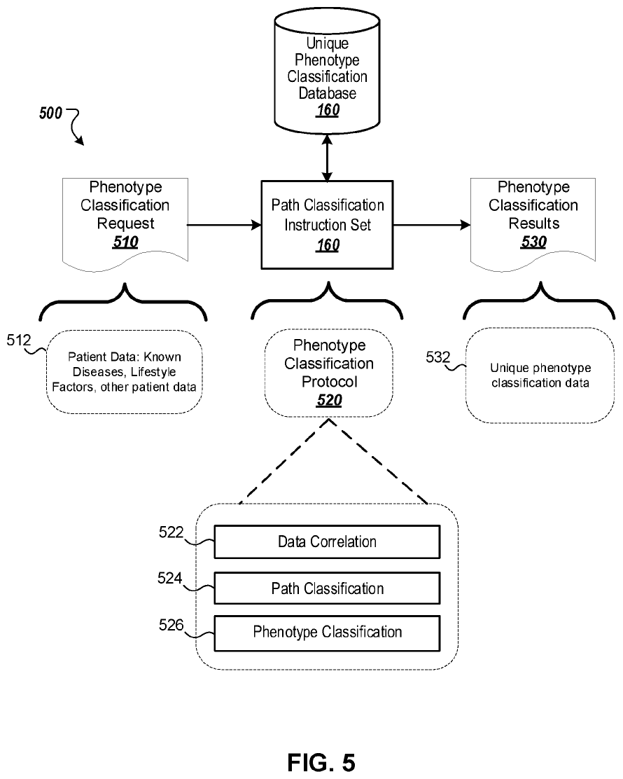

FIG. 5 illustrates an example phenotype classification process based on a phenotype classification request, according to embodiments of the invention. In particular, FIG. 5 illustrates an example environment 500 for a phenotype classification implementation for determining phenotype classification results 530 based on receiving a phenotype classification request 510. The objective for the phenotype classification instruction set is to enable healthcare personnel (e.g., physicians, scientists, etc.) to inform immediate care, future care, and the advancement of medical research, in an industry applicable, fully automated manner. Additionally, the phenotype classification process enables healthcare providers to provide a clinician with the possibility of determining a unique patient phenotype classification for a particular patient that can be used for clinical research and/or treatment reference. As the machine learning model acquires more patient data, database data tables increase as more path classification outcomes are added. For example, outcomes are identified at time of detection and identification of risk.

In an exemplary implementation of the invention, the phenotype classification instruction set 160, stored on phenotype classification server 140, receives a phenotype classification request 510 (e.g., from a healthcare entity via a client device 110). The phenotype classification request 510 includes phenotype classification request information 512 (e.g., patient data, i.e., known diseases, lifestyle factors, and other patient data) that is associated with a phenotype classification for a patient. The phenotype classification instruction set 160 initiates a phenotype classification protocol 520 to generate phenotype classification results 532. The phenotype classification protocol 520 includes, for example, a data correlation module 522 (e.g., data correlator module 152), a path classification module 524 (e.g., path classification module 154), and a phenotype classification module 526 (e.g., phenotype classification module 156). The phenotype classification results 532 sent to the requestor may include unique phenotype classification data (e.g., a unique sequenced protocol associated with the particular patient at that time of the request).

FIG. 6 illustrates a flowchart of an example process 600 for implementing a phenotype classification process using machine learning models, according to embodiments of the invention. Operations of the process 600 can be implemented, for example, by a system that includes one or more data processing apparatus, such as the phenotype classification server 140 of FIG. 1. The process 600 can also be implemented by instructions (e.g., phenotype classification instruction set 150) stored on computer storage medium, where execution of the instructions by a system that includes a data processing apparatus cause the data processing apparatus to perform the operations of the process 600. A machine-learned model (e.g., machine learning module 158) is trained based on a patient data classification path process for a plurality of iterations, where each iteration follows the process 600 as described herein.

The system obtains patient data stored within a patient databases (610). For example, data correlation (e.g., via data correlator module 152) may include analyzing the patient records for patient's real time data inputs that are captured based on data tables that include various population health parameters. Data inputs are collected manually, automatically via devices, and by geolocation. Patient data input prompts may be automated based on certain general health and health determinants factors. The patient data elements may include a type of disease and date of contraction (if known, lifestyle factors, other factors including administrative and demographic information, diagnosis, treatment, prescription drugs, laboratory tests, physiologic monitoring data).

The system evaluates the patient data elements to determine and identify classification results based on predetermined classification database tables (620) and determines patient data classification path features based on the identified classification results (630). For example, the system (e.g., via path classification module 154) may evaluate the patient data elements to determine and identify classification results based on predetermined classification database tables based on the received correlated data from the data correlator module 152. In some implementations of the invention, the path classification module 154 may determine patient data classification path features based on the identified classification results. For example, classification results determine paths, and each path classification may have a set of time sequenced data inputs and correlation analysis that trigger real time tracking instances for continuous monitoring, preventing, and detecting diseases that may be associated with a specific path classification. Additionally, within each path classification there may be a specific set of unique timed instances.

The system selects one or more of the patient data classification path features for inclusion in the machine-learned patient data classification path process using a sequencing protocol that defines a minimal causal relationship that exists between a particular patient data classification path feature and identified patterns (640). For example, the system (e.g., via phenotype classification module 156) may utilize the outcomes of a patient classification that were correlated to determine a unique user phenotype classification. The unique user phenotype classification may be determined by a timeline of risk and detection of a disease based on the patient's individual health status (e.g., contraction of a disease and interrelated diseases, timeframe of the contraction of a disease, or not contracting the disease at all, and the like). For example, some diseases may be interrelated, but only for a particular period of time. For example, an active phase of disease A (e.g., 10-year active phase contracted in 2018) may affect the health/phenotype classification of a patient who also has disease B (e.g., 15-year active phase contracted in 2010) during an overlapping active phase (e.g., overlaps for seven years from year 2018 to 2025).

In some implementations of the invention, the machine-learned patient data classification path process is based on determining a timeline of risk and detection of disease based on a patient's individual health status. For example, the timeline of risk and the detection of disease based on a patient's individual health status may be based on a contraction of a disease and interrelated diseases, a timeframe of the contraction of a disease, or not contracting the disease at all.

In some implementations of the invention, the minimal causal relationship exists before that particular patient data classification path feature is included in the machine-learned patient data classification path process.

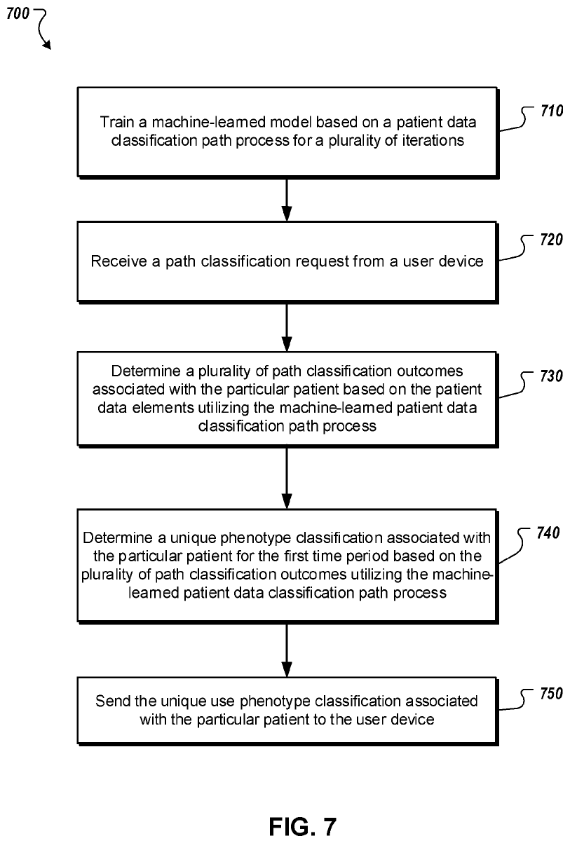

FIG. 7 illustrates a flowchart of an example process 700 for implementing a phenotype classification process using machine learning models, according to embodiments of the invention. Operations of the process 700 can be implemented, for example, by a system that includes one or more data processing apparatus, such as the phenotype classification server 140 of FIG. 1. The process 700 can also be implemented by instructions (e.g., phenotype classification instruction set 150) stored on computer storage medium, where execution of the instructions by a system that includes a data processing apparatus cause the data processing apparatus to perform the operations of the process 700.

The system trains a machine-learned model based on a patient data classification path process for a plurality of iterations (710). For example, as discussed herein with reference to process 600, a machine-learned model is trained based on a patient data classification path process for a plurality of iterations. For example, the system obtains patient data stored within a patient databases (610), evaluates the patient data elements to determine and identify classification results based on predetermined classification database tables (620), determines patient data classification path features based on the identified classification results (630), and selects one or more of the patient data classification path features for inclusion in the machine-learned patient data classification path process using a sequencing protocol that defines a minimal causal relationship that exists between a particular patient data classification path feature and identified patterns (640). The machine learning model may be carried out continuously, periodically and/or on-demand in order to maintain currency of the machine learning model based on real time patient data inputs that are obtained.

The system receives a path classification request from a user device (720). In some implementations of the invention, the path classification request includes a first set of patient data elements associated with a particular patient for a first time period. For example, the first set of patient data elements may include diseases known, time frames/dates, lifestyle factors, and the like. In some implementations of the invention, the patient data elements associated with the particular patient includes a first disease that includes an active time window. In some implementations of the invention, the patient data elements associated with the particular patient includes a type of disease and a date of contraction.

The system determines a plurality of path classification outcomes associated with the particular patient based on the patient data elements (730). In some implementations of the invention, a machine-learned patient data classification path process using the machine learning model that trained for process 600 is utilized. For example, the phenotype classification instruction set 150 may utilize the paths classifications data and correlate and sequence the data in real time to determine unique phenotype outcomes. A unique phenotype classification associated with the particular patient for the first time period may be determined utilizing the machine-learned patient data classification path process and based on the plurality of path classification outcomes.

The system determines a unique phenotype classification associated with the particular patient for the first time period based on the plurality of path classification outcomes (740). In some implementations of the invention, a machine-learned patient data classification path process using the machine learning model that trained for process 600 is utilized. For example, user phenotype classification is correlated in phenotype classification database for clinical research and/or treatment reference.

The system sends the unique user phenotype classification associated with the particular patient to the user device (750). For example, after the phenotype classification instruction set 150 determines a unique user phenotype classification associated with the particular patient from the initial request, the phenotype classification server 140 can then send the results to the requesting device (e.g., user device 110).

In some implementations of the invention, determining the unique phenotype classification associated with the particular patient for the first time period is based on detecting a disease that is associated with the unique phenotype classification associated with the particular patient. For example, within each path classification there is a specific set of unique timed instances.

In some implementations of the invention, process 700 further includes receiving a second path classification request from the user device, the second path classification request including a second set of patient data elements associated with the particular patient for a second time period, and determining a second phenotype classification associated with the particular patient for the second time period. In some implementations of the invention, the second phenotype classification is different than the first phenotype classification. For example, a time sequence automates a year-over-year monitoring. In some implementations of the invention, the first set of patient data elements includes a first disease, and the second set of patient data elements includes a second disease that is different than the first disease, wherein the first disease and second disease include interrelated attributes. For example, a first disease and a second disease may each have different time windows of being active, but when both are active at the same time the first disease and the second disease can react and provide different clinical outcomes based on the respective active time windows. In some implementations of the invention, determining the second phenotype classification associated with the particular patient for the second time period is based on analysis of an active time window associated with the first disease and an active time window associated with the second disease (e.g., based on an analysis of interrelated diseases, and different time windows of being active.

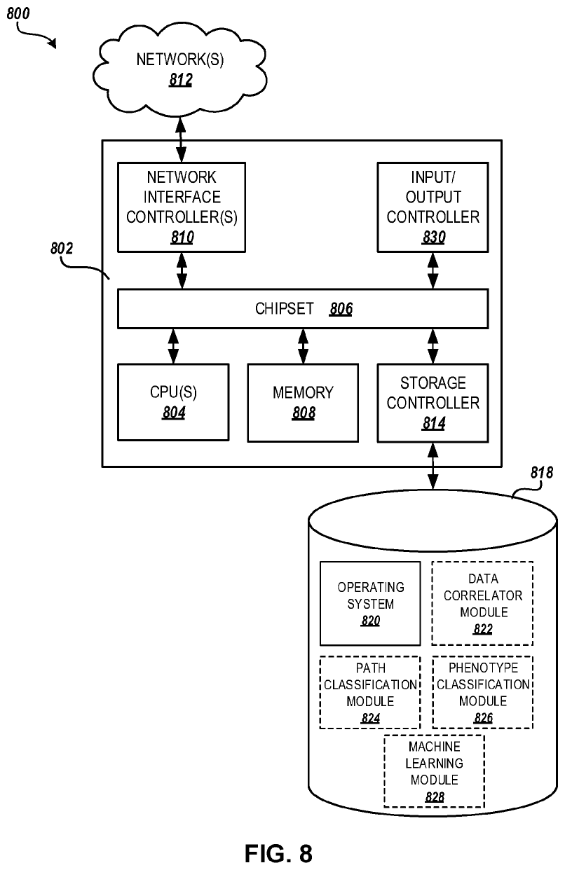

FIG. 8 illustrates an example computer architecture 800 for a computer 802 capable of executing the software components described herein for the sending/receiving and processing of tasks. The computer architecture 800 (also referred to herein as a “server”) shown in FIG. 8 illustrates a server computer, workstation, desktop computer, laptop, a server operating in a cloud environment, or other computing device, and may be utilized to execute any aspects of the software components presented herein described as executing on a host server, or other computing platform. The computer 802 preferably includes a baseboard, or “motherboard,” which is a printed circuit board to which a multitude of components or devices may be connected by way of a system bus or other electrical communication paths. In one illustrative embodiment, one or more central processing units (CPUs) 804 operate in conjunction with a chipset 806. The CPUs 804 can be programmable processors that perform arithmetic and logical operations necessary for the operation of the computer 802.

The CPUs 804 preferably perform operations by transitioning from one discrete, physical state to the next through the manipulation of switching elements that differentiate between and change these states. Switching elements may generally include electronic circuits that maintain one of two binary states, such as flip-flops, and electronic circuits that provide an output state based on the logical combination of the states of one or more other switching elements, such as logic gates. These basic switching elements may be combined to create more complex logic circuits, including registers, adders-subtractors, arithmetic logic units, floating-point units, or the like.

The chipset 806 provides an interface between the CPUs 804 and the remainder of the components and devices on the baseboard. The chipset 806 may provide an interface to a memory 808. The memory 808 may include a random-access memory (RAM) used as the main memory in the computer 802. The memory 808 may further include a computer-readable storage medium such as a read-only memory (ROM) or non-volatile RAM (NVRAM) for storing basic routines that help to startup the computer 802 and to transfer information between the various components and devices. The ROM or NVRAM may also store other software components necessary for the operation of the computer 802 in accordance with the embodiments described herein.

According to various embodiments, the computer 802 may operate in a networked environment using logical connections to remote computing devices through one or more networks 812, a local-area network (LAN), a wide-area network (WAN), the Internet, or any other networking topology known in the art that connects the computer 802 to the devices and other remote computers. The chipset 806 includes functionality for providing network connectivity through one or more network interface controllers (NICs) 810, such as a gigabit Ethernet adapter. For example, the NIC 810 may be capable of connecting the computer 802 to other computer devices in the utility provider's systems. It should be appreciated that any number of NICs 810 may be present in the computer 802, connecting the computer to other types of networks and remote computer systems beyond those described herein.

The computer 802 may be connected to at least one mass storage device 818 that provides non-volatile storage for the computer 802. The mass storage device 818 may store system programs, application programs, other program modules, and data, which are described in greater detail herein. The mass storage device 818 may be connected to the computer 802 through a storage controller 814 connected to the chipset 806. The mass storage device 818 may consist of one or more physical storage units. The storage controller 814 may interface with the physical storage units through a serial attached SCSI (SAS) interface, a serial advanced technology attachment (SATA) interface, a fiber channel (FC) interface, or other standard interface for physically connecting and transferring data between computers and physical storage devices.

The computer 802 may store data on the mass storage device 818 by transforming the physical state of the physical storage units to reflect the information being stored. The specific transformation of physical state may depend on various factors, in different embodiments of the invention of this description. Examples of such factors may include, but are not limited to, the technology used to implement the physical storage units, whether the mass storage device 818 is characterized as primary or secondary storage, or the like. For example, the computer 802 may store information to the mass storage device 818 by issuing instructions through the storage controller 814 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computer 802 may further read information from the mass storage device 818 by detecting the physical states or characteristics of one or more particular locations within the physical storage units.

The mass storage device 818 may store an operating system 820 utilized to control the operation of the computer 802. According to some embodiments, the operating system includes the LINUX operating system. According to another embodiment, the operating system includes the WINDOWS®SERVER operating system from MICROSOFT Corporation of Redmond, Wash. According to further embodiments, the operating system may include the UNIX or SOLARIS operating systems. It should be appreciated that other operating systems may also be utilized. The mass storage device 818 may store other system or application programs and data utilized by the computer 802, such as data correlator module 822 to perform the data correlation processes, a path classification module 824 to perform the path classification processes, a phenotype classification module 826 to perform the phenotype classification processes, and a machine learning module 828, according to embodiments described herein. Other system or application programs and data utilized by the computer 802 may be provided as well (e.g., a security module, a payment processing module, a user interface module, etc.).

In some embodiments, the mass storage device 818 may be encoded with computer-executable instructions that, when loaded into the computer 802, transforms the computer 802 from being a general-purpose computing system into a special-purpose computer capable of implementing the embodiments described herein. These computer-executable instructions transform the computer 802 by specifying how the CPUs 804 transition between states, as described above. According to some embodiments, from the phenotype classification server 140 perspective, the mass storage device 818 stores computer-executable instructions that, when executed by the computer 802, perform portions of the process 600, for training a machine learning model, and perform portions of the process 700, for implementing a phenotype classification system, as described herein. In further embodiments, the computer 802 may have access to other computer-readable storage medium in addition to or as an alternative to the mass storage device 818.

The computer 802 may also include an input/output controller 830 for receiving and processing input from a number of input devices, such as a keyboard, a mouse, a touchpad, a touch screen, an electronic stylus, or other type of input device. Similarly, the input/output controller 830 may provide output to a display device, such as a computer monitor, a flat-panel display, a digital projector, a printer, a plotter, or other type of output device. It will be appreciated that the computer 802 may not include all of the components shown in FIG. 8, may include other components that are not explicitly shown in FIG. 8, or may utilize an architecture completely different than that shown in FIG. 8.

In general, the routines executed to implement the embodiments of the invention, whether implemented as part of an operating system or a specific application, component, program, object, module or sequence of instructions, or even a subset thereof, may be referred to herein as “computer program code,” or simply “program code.” Program code typically includes computer readable instructions that are resident at various times in various memory and storage devices in a computer and that, when read and executed by one or more processors in a computer, cause that computer to perform the operations necessary to execute operations and/or elements embodying the various aspects of the embodiments of the invention. Computer readable program instructions for carrying out operations of the embodiments of the invention may be, for example, assembly language or either source code or object code written in any combination of one or more programming languages.

The program code embodied in any of the applications/modules described herein is capable of being individually or collectively distributed as a program product in a variety of different forms. In particular, the program code may be distributed using a computer readable storage medium having computer readable program instructions thereon for causing a processor to carry out aspects of the embodiments of the invention.

Computer readable storage media, which is inherently non-transitory, may include volatile and non-volatile, and removable and non-removable tangible media implemented in any method or technology for storage of information, such as computer-readable instructions, data structures, program modules, or other data. Computer readable storage media may further include random access memory (RAM), read-only memory (ROM), erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. A computer readable storage medium should not be construed as transitory signals per se (e.g., radio waves or other propagating electromagnetic waves, electromagnetic waves propagating through a transmission media such as a waveguide, or electrical signals transmitted through a wire). Computer readable program instructions may be downloaded to a computer, another type of programmable data processing apparatus, or another device from a computer readable storage medium or to an external computer or external storage device via a network.

Computer readable program instructions stored in a computer readable medium may be used to direct a computer, other types of programmable data processing apparatus, or other devices to function in a particular manner, such that the instructions stored in the computer readable medium produce an article of manufacture including instructions that implement the functions/acts specified in the flowcharts, sequence diagrams, and/or block diagrams. The computer program instructions may be provided to one or more processors of a general purpose computer, special purpose computer, or other programmable data processing apparatus to produce a machine, such that the instructions, which execute via the one or more processors, cause a series of computations to be performed to implement the functions and/or acts specified in the flowcharts, sequence diagrams, and/or block diagrams.

In certain alternative embodiments, the functions and/or acts specified in the flowcharts, sequence diagrams, and/or block diagrams may be re-ordered, processed serially, and/or processed concurrently without departing from the scope of the embodiments of the invention. Moreover, any of the flowcharts, sequence diagrams, and/or block diagrams may include more or fewer blocks than those illustrated consistent with embodiments of the invention.

The terminology used herein is for the purpose of describing particular embodiments only and is not intended to be limiting of the embodiments of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “includes”, “having”, “has”, “with”, “comprised of”, or variants thereof are used in either the detailed description or the claims, such terms are intended to be inclusive in a manner similar to the term “comprising.”

While all of the invention has been illustrated by a description of various embodiments and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

1-33. (canceled)

34. A method comprising:
at an electronic device having a processor:
training a machine-learned model based on a patient data classification path process for each iteration of a plurality of iterations by:
obtaining patient data stored within a patient database, wherein the patient database is populated with a plurality of patient data elements associated with one or more patients;
evaluating the patient data elements to determine and identify classification results based on predetermined classification database tables;
determining a plurality of patient data classification path features based on the identified classification results; and
selecting one or more of the patient data classification path features for inclusion in a machine-learned patient data classification path process using a sequencing protocol that defines a minimal causal relationship that exists between a particular patient data classification path feature and identified patterns;
receiving a phenotype classification request from a user device, wherein the phenotype classification request comprises a first set of patient data elements associated with a particular patient for a first time period;
determining, utilizing the machine-learned patient data classification path process, a plurality of path classification outcomes associated with the particular patient based on the patient data elements; and
determining, utilizing the machine-learned patient data classification path process, a unique phenotype classification associated with the particular patient for the first time period based on the plurality of path classification outcomes.

35. The method of claim 34 wherein the patient data elements associated with the particular patient comprises a first disease that includes an active time window.

36. The method of claim 34 wherein the patient data elements associated with the particular patient comprises a type of disease and a date of contraction.

37. The method of claim 34 further comprising:
sending the unique use phenotype classification associated with the particular patient to the user device.

38. The method of claim 34 wherein determining the unique phenotype classification associated with the particular patient for the first time period is based on detecting a disease that is associated with the unique phenotype classification associated with the particular patient.

39. The method of claim 34 further comprising:
receiving a second path classification request from the user device, the second path classification request comprising a second set of patient data elements associated with the particular patient for a second time period; and
determining a second phenotype classification associated with the particular patient for the second time period.

40. The method of claim 39 wherein the first set of patient data elements comprises a first disease, the second set of patient data elements comprises a second disease that is different than the first disease, and the first disease and the second disease comprise interrelated attributes.

41. The method of claim 40 wherein determining the second phenotype classification associated with the particular patient for the second time period is based on analysis of a first active time window associated with the first disease and a second active time window associated with the second disease.

42. The method of claim 39 wherein the unique phenotype classification is a first phenotype classification, and the second phenotype classification is different than the first phenotype classification.

43. The method of claim 34 wherein the machine-learned patient data classification path process is based on determining a timeline of risk and detection of disease based on a patient's individual health status.

44. The method of claim 34 wherein the minimal causal relationship exists before that particular patient data classification path feature is included in the machine-learned patient data classification path process.

45. A computing apparatus comprising:
one or more processors;
at least one memory device coupled with the one or more processors; and
a data communications interface operably associated with the one or more processors,
wherein the at least one memory device contains a plurality of program instructions that, when executed by the one or more processors, cause the computing apparatus to:
train a machine-learned model based on a patient data classification path process for each iteration of a plurality of iterations by:
obtaining patient data stored within a patient database, wherein the patient database is populated with a plurality of patient data elements associated with one or more patients;
evaluating the patient data elements to determine and identify classification results based on predetermined classification database tables;
determining a plurality of patient data classification path features based on the identified classification results; and
selecting one or more of the patient data classification path features for inclusion in a machine-learned patient data classification path process using a sequencing protocol that defines a minimal causal relationship that exists between a particular patient data classification path feature and identified patterns;
receive a phenotype classification request from a user device, wherein the phenotype classification request comprises a first set of patient data elements associated with a particular patient for a first time period;
determine, utilizing the machine-learned patient data classification path process, a plurality of path classification outcomes associated with the particular patient based on the patient data elements; and
determine, utilizing the machine-learned patient data classification path process, a unique phenotype classification associated with the particular patient for the first time period based on the plurality of path classification outcomes.

46. The computing apparatus of claim 45 wherein the patient data elements associated with the particular patient comprises a first disease that includes an active time window.

47. The computing apparatus of claim 45 wherein the patient data elements associated with the particular patient comprises a type of disease and a date of contraction.

48. The computing apparatus of claim 45 wherein the plurality of program instructions that, when executed by the one or more processors, further cause the computing apparatus to:
send the unique use phenotype classification associated with the particular patient to the user device.

49. The computing apparatus of claim 45 wherein determine the unique phenotype classification associated with the particular patient for the first time period is based on detecting a disease that is associated with the unique phenotype classification associated with the particular patient.

50. The computing apparatus of claim 45 wherein the plurality of program instructions that, when executed by the one or more processors, further cause the computing apparatus to:
receive a second path classification request from the user device, the second path classification request comprising a second set of patient data elements associated with the particular patient for a second time period; and
determine a second phenotype classification associated with the particular patient for the second time period.

51. The computing apparatus of claim 50 wherein the first set of patient data elements comprises a first disease, the second set of patient data elements comprises a second disease that is different than the first disease, and the first disease and the second disease comprise interrelated attributes.

52. The computing apparatus of claim 51 wherein determining the second phenotype classification associated with the particular patient for the second time period is based on analysis of a first active time window associated with the first disease and a second active time window associated with the second disease.

53. A non-transitory computer storage medium encoded with a computer program, the computer program comprising a plurality of program instructions that when executed by one or more processors cause the one or more processors to perform operations comprising:
train a machine-learned model based on a patient data classification path process for each iteration of a plurality of iterations by:
obtaining patient data stored within a patient database, wherein the patient database is populated with a plurality of patient data elements associated with one or more patients;
evaluating the patient data elements to determine and identify classification results based on predetermined classification database tables;
determining a plurality of patient data classification path features based on the identified classification results; and
selecting one or more of the patient data classification path features for inclusion in a machine-learned patient data classification path process using a sequencing protocol that defines a minimal causal relationship that exists between a particular patient data classification path feature and identified patterns;
receive a phenotype classification request from a user device, wherein the phenotype classification request comprises a first set patient data elements associated with a particular patient for a first time period;
determine, utilizing the machine-learned patient data classification path process, a plurality of path classification outcomes associated with the particular patient based on the patient data elements; and
determine, utilizing the machine-learned patient data classification path process, a unique phenotype classification associated with the particular patient for the first time period based on the plurality of path classification outcomes.
